(19) United States
(12) Patent Application Publication
(10) Pub. No.: US 2013/0236399 A1
(43) Pub. Date: Sep. 12, 2013

(54) APATITE COMPOSITIONS

(71) Applicants: Willem Frederik van den Bosch, Oegstgeest (NL); Raimond Nicolaas Bruno Van Duinen, The Hague (NL)

(72) Inventors: Willem Frederik van den Bosch, Oegstgeest (NL); Raimond Nicolaas Bruno Van Duinen, The Hague (NL)

(73) Assignee: Stitching Glass For Health

(21) Appl. No.: 13/809,396

(22) PCT Filed: Jul. 8, 2011
(86) PCT No.: PCT/NL2011/050501

§ 371 Date: May 28, 2013

Related U.S. Application Data
(60) Provisional application No. 61/362,859, filed on Jul. 9, 2010.

(51) Int. Cl. A61K 8/897; A61K 6/033; A61L 27/12; A61K 8/24; A61K 6/00; A61L 27/18; A61Q 11/00; A61K 6/093

(52) U.S. Cl. CPC A61K 8/897; A61Q 11/00; A61K 6/033; A61K 6/093; A61K 8/24; A61K 6/0023; A61L 27/18; A61L 27/12
USPC 424/52; 523/116

(57) ABSTRACT

The present invention relates to an apatite composition comprising 0.001-99.999 wt. % of an apatite and 0.001-99.999 wt. % of a glass carbomer, based on the total weight of the apatite composition. The apatite is preferably a fluoroapatite according to the general formula (I): wherein: M is a cation other than Ca²⁺, B is an anion other than P04³″, A is F″, 0≦x≦9, 0≦y≦5, and 0≦z≦2, and most preferably Ca₁₀(PO₄)₆F₂. The apatite composition is advantageously used as a dental filling material, a denting bonding cement, a bone cement, a bone replacing material, in dental care and cleansing products, glass-ceramics, bone scaffolds and bone substrates, and as a coating material for crowns and bridges.

### FIELD OF THE INVENTION

The present application relates to an apatite composition and in particular to a fluoroapatite composition and the use of these compositions in dental and medical applications.

### BACKGROUND OF THE INVENTION

Compositions comprising apatite materials are often used for medical and dental purposes and may also be used in combinations with other materials such as polymers, glasses and the like. Important properties which are partly dependent from the intended application of such compositions are transparency, adhesion, remineralisation stimulation, hardness, curability and polishability. U.S. Pat. No. 5,952,399, which is incorporated by reference, for example, discloses a dental composition that would have such properties.

WO 20040108095, incorporated by reference, discloses a self hardening glass carbomer composition obtainable by treating a fluorosilicate glass powder with a poly(dialkylsiloxane) having terminal hydroxyl groups, wherein the alkyl groups contain 1 to 4 carbon atoms, and subsequently with an aqueous acid solution, followed by separating the treated fluorosilicate glass powder from the aqueous acid solution. The self hardening glass carbomer composition is used as a dental filling material, as a denting bonding cement, as a bone cement or as a bone replacing material.

WO 2005110339, incorporated by reference, discloses a composition containing nano-crystalline apatite that is used for restoring bone and teeth, said composition comprising a mixture of a crosslinkable resin and/or a hardenable acid/base cement system, an apatite and optionally a filler. The apatite may be a fluoroapatite or a hydroxyapatite. The hardenable acid/base cement system may be a dental polyalkanoate cement comprising fluoroaluminosilicate glass powder or a metal oxide, e.g. zinc oxide or magnesium oxide, a crosslinking polyalkenoic acid and water.

WO2007051543, incorporated by reference, discloses an oral and dental care agent and a dental cleaning agent comprising hydroxyapatite, fluoroapatite or calciumfluoride.

Other dental care and cleansing compositions comprising fluoroapatite are for example disclosed in WO 2000037033, WO 2005027863, WO 2007051543, WO 2007051544, WO 2007051546, all incorporated by reference herein. Dental filling materials comprising fluoroapatite are for example disclosed in WO 2004058207, WO 2007051547 and WO 2007051542, all incorporated by reference.

The use of fluoroapatite as a bonding cement for adhering hydroxyapatite to zirconiumoxide bone scaffolds is for example disclosed by Kim et al., Biomaterials 24(19), 3277-3284, 2003, Biomaterials, 25(15), 2919-2926, 2004 and J. Biomater. Appl. 22(6), 484-504, 2008, both incorporated by reference.

However, there is still a need for compositions having improved properties, in particular transparency, adhesion, remineralisation stimulation, hardness, curability and polishability as disclosed above. One of the most important advantages of the apatite composition and in particular of the fluoroapatite composition according to the present invention is that they provide improved bone remineralisation and adhesion to bone, in particular teeth.

### SUMMARY OF THE INVENTION

The present invention relates to an apatite composition comprising 0.001-99.999 wt. % of an apatite and 0.001-99.999 wt. % of a glass carbomer, based on the total weight of the apatite composition, and the use thereof. The present invention further relates to the use of the apatite composition in dental and bone applications, in particular as a dental filling material, a dental coating material, a dental bonding cement, a bone cement and a bone replacing material. The apatite composition can further advantageously inter alia be used in dental care and cleansing products, glass-ceramics, bone scaffolds and bone substrates.

### DETAILED DESCRIPTION OF THE INVENTION

### Definitions

The verb “to comprise” as is used in this description and in the claims and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there is one and only one of the elements. The indefinite article “a” or “an” thus usually means “at least one”.

### The Glass Carbomer

The glass carbomer and its manufacture is disclosed in WO 20040108095. This material is manufactured from commonly available materials and it shows—either in the unhardened state or the hardened state—a better performance when compared to glass ionomers known from the prior art. The glass carbomer has for example good toughness and strength and excellent fluoride release. In addition, the glass carbomer does not show shrinkage or expansion, an essential property for providing fillings for cavities having high strength and long durability.

In addition, the glass carbomer has—when hardened—in particular a higher hardness (i.e. a higher flexural and compressive strength), a lower sensitivity towards abrasion and wear, a greater stiffness, a lower solubility, a smoother surface, a better colourfastness, a better adherence to e.g. bone tissue and a lower water sensitivity. Another advantage of the glass carbomer is that when hardened it can be polished much easier in comparison with known glass ionomers. A further advantage of the glass carbomer is that the unhardened glass carbomer shows a better fluidity so that cavities are much easier to fill, a better processability and a shorter hardening time. The glass carbomer is also much easier to use as sealing material.

The glass carbomer is obtainable by:
(a) treating a fluorosilicate glass powder with a poly(dialkylsiloxane) having terminal hydroxyl groups, wherein the alkyl groups contain 1 to 4 carbon atoms, to form a first treated fluorosilicate glass,
(b) treating the treated fluorosilicate glass with an aqueous acid solution to form a second treated fluorosilicate glass which treated, and
(c) separating the second treated fluorosilicate glass powder from the aqueous acid solution.

Alternatively, the glass carbomer is obtainable by treating a fluorosilicate glass powder subsequently with: (a) a poly(dialkylsiloxane) having terminal hydroxyl groups, wherein the alkyl groups contain 1 to 4 carbon atoms and (b) an aqueous acid solution, followed by separating the treated fluorosilicate glass powder from the aqueous acid solution.

The poly(dialkylsiloxane) used in step (a) may be linear or cyclic. It may further be a blend of different poly(dialkylsiloxane)s, e.g. a blend of a poly(dimethylsioxane) of high kinematic viscosity and a poly(dimethylsiloxane) of low kinematic viscosity. It is further preferred that the alkyl groups of the poly(dialkylsiloxane) are methyl groups. The kinematic viscosity is preferably in the range of about 1 cSt to about 100.000 cSt at 25° C. [about 1 to about 100.000 mm²/s], preferably about 100 cSt to about 10.000 cSt at 25° C. [about 100 to about 10.000 mm²/s], even more preferably about 500 cSt to about 5000 cSt at 25° C. [about 500 to about 5.000 mm²/s]. The best results are obtained with a viscosity of about 1000 cSt at 25° C. [about 1000 mm²/s].

The aqueous acid solution used in step (b) comprises an inorganic acid or an organic acid. It is preferred that the aqueous acid solution comprises an organic acid, wherein the organic acid is preferably a polymer, e.g. a polyacrylic acid. According to the invention, the aqueous acid solution has a pH in the range of about 2 to about 7.

The fluorosilicate glass powder consists of particles which are depleted of calcium at their surface such that the quotient of the atomic ratio Si/Ca at the surface of the particles and the atomic ratio Si/Ca in the core region is at least about 2.0, preferably at least about 3.0, and most preferably at least about 4.0. The calcium content of the particles increases asymptotically from the surface to the core region.

The depth of the depletion zone depends on the conditions given in each individual case. However, the depletion zone preferably extends at least to a depth of about 10 nm, more preferably to at least about 20 nm, and most preferably to at least about 100 nm. These ranges are particularly suited for use of the fluorosilicate glass powders in dentistry. For other purposes, e.g., for use in bone cements, the depletion zone may also be deeper and may be 200 to 300 nm.

The surface measurement to determine Ca depletion of the glass powders of the invention is suitably carried out by photo electron spectroscopy for chemical analysis (ESCA; cf. for example R. S. Swingle II and W. M. Riggs in Critical Reviews in Analytical Chemistry, Vol. 5, Issue 3, pages 267 to 321, 1975 and K. Levsen in “Chemie in unserer Zeit”, Vol. 40, pages 48 to 53, 1976).

The particles of the fluorosilicate glass powder have preferably an average particle size (weight average) of about 10⁻³ μm to about 200 μm, more preferably about 2×10⁻³ μm to about 200 μm, even more preferably about 5×10⁻² μm to about 150 μm, yet even more preferably about 0.1 μm to about 100 μm and in particular about 0.1 μm to about 80 μm. Most preferably, the average particle size does not exceed about 50 μm.

More in particular, the particles of the fluorosilicate glass powder have an average particle size (weight average) of at least about 10⁻³ μm, preferably at least about 2×10⁻³ μm, more preferably at least about 10⁻² μm, and most preferably at least about 0.1 μm. For dental purposes the average particle size (weight average) is about 10⁻³ μm to about 20.0 μm, preferably about 2×10⁻³ to about 20.0 μm, more preferably about 3.0 to about 15.0 μm, most preferably about 3.0 to about 10.0 μm. The particles have a maximum particle size of about 200 μm, more preferably about 150 μm, even more preferably about 100 μm, yet even more preferably about 80 μm and especially about 50 μm. For use as dental bonding cement the maximum particle size is about 25 μm, preferably about 20 μm. In order to achieve good mechanical properties, a not excessively narrow particle size distribution is favourable, as usual, which is achieved, for example, by conventional grinding and classifying of the coarse particles.

The fluorosilicate glass powder is prepared from a glass powder having the average composition of the core region of the glass carbomer. Suitable glass powders are disclosed in for example GB A 1.316.129, Table 1. The glass powders employed as starting materials are obtained as usual by fusing the starting components together at temperatures above about 950° C., quenching, and grinding.

The thus obtained powders are then subjected to a surface treatment. The powders are obtainable, for example, by removal of Ca by suitable chemical agents.

For example, the starting glass powders are treated on the surface with an acid, preferably at room temperature. To this end, substances containing acidic groups are employed, preferably substances forming soluble calcium salts. The reaction period varies between a few minutes and several days, depending on the strength and concentration of the acid employed. As acids, organic acids or inorganic acids such as hydrochloric acid, sulphuric acid, nitric acid, acetic acid, propionic acid and perchloric acid may be used.

The acids are preferably employed at a concentration of about 0.01 to about 10% by weight, preferably from about 0.05 to about 3% by weight, relative to the weight of the glass powder.

The powders are then separated from the acidic solution and thoroughly washed to leave substantially no soluble calcium salts on the surface of the powder particles. Finally the powder is dried, preferably above about 70° C., and screened to the desired particle size ranges.

The stronger the acid employed and the longer a given acid acts on the powder the longer will be the processing period after mixing with the mixing fluid.

The glass carbomer is preferably not resin-modified or resin-reinforced since such materials have the disadvantage that carcinogenic or toxic components often leach out.

### The Apatite

The apatite used in the apatite composition according to the present invention can be of any kind including hydroxyapatite, fluoride-doped hydroxyapatatite and fluoroapatite and mixtures thereof. It is well known that apatites can have different molecular formulas, see for example U.S. Pat. No. 5,952,399, incorporated by reference herein.

It is preferred that the apatite is a fluoroapatite, in particular a fluoroapatite according to general formula (I):

Ca₁₀₋ₓMₓ(PO₄)_{6-y}B_{y}A_{z}(OH)_{2-z} (I)

wherein M is a cation other than Ca²⁺, B is an anion other than PO₄³⁻; A is F⁻; 0≦x≦9; 0≦y≦5; and 0≦z≦2. Most preferably, the apatite is a fluoroapatite according to the general formula (I) wherein x=y=0 and z=2, i.e. a compound according to the formula Ca₁₀(PO₄)₆F₂ (fluoroapatite).

The apatite may comprise a mixture of different apatites, e.g. a mixture of Ca₁₀(PO₄)₆F₂ (fluoroapatite) and one or more apatites according to the general formula (I), wherein M, B, x, y and z are defined as above and wherein A is selected from the group consisting of O²⁻, CO₃²⁻, Cl⁻, OH⁻ and mixtures thereof, preferably O²⁻, CO₃²⁻, OH⁻ and mixtures thereof. In particular, if the apatite is a mixture, it is preferably a mixture of Ca₁₀(PO₄)₆F₂ (fluoroapatite) and Ca₁₀(PO₄)₆(OH)₂ (hydroxyapatite; i.e. that in the general formula (I) x=y=z=0 and A is OH⁻), wherein it is preferred that the weight ratio of Ca₁₀(PO₄)₆F₂ (fluoroapatite) and Ca₁₀(PO₄)₆(OH)₂ (hydroxyapatite) ranges from 99:1 to 1:99, more preferably 90:1 to 1:90, based on the total weight of the apatite mixture.

Additionally, the apatite may be surface treated, in particular with esters of phosphoric acids, phosphonic acids, carboxylic acids and mixtures thereof.

### The Apatite Composition

According to the invention, the apatite composition comprises 0.001-99.999 wt. % of an apatite and 0.001-99.999 wt. % of a glass carbomer, based on the total weight of the apatite composition. More preferably, the apatite composition comprises 1-99 wt. % of an apatite and 1-99 wt. % of a glass carbomer, based on the total weight of the apatite composition. Even more preferably, the apatite composition comprises 10-90 wt. % of an apatite and 10-90 wt. % of a glass carbomer, based on the total weight of the apatite composition. The apatite compositions are preferably amorphous.

The apatite composition may comprise further components, e.g. filler materials like powder and silica, glass-ceramic materials, glasses, microbeads, inorganic fibres, organic fibres and mixtures thereof. If the apatite composition comprises a filler material, the filler material is preferably presenting an amount of about 0.1 to about 90% by weight, more preferably about 1 to about 90% by weight, based on the total weight of the apatite composition. It is also preferred that the filler material has a refractive index of about 1.45 to about 1.54 so that the apatite composition comprising the filler material has good aesthetics and has an improved transparency. The filler materials preferably have an average particle size in the range of 10⁻³ μm to about 200 μm, more preferably about 2×10⁻³ μm to about 200 μm, even more preferably 5×10⁻² μm to about 150 μm, yet even more preferably about 0.1 μm to about 100 μm, yet even more preferably about 0.1 μm to about 80 μm and in particular about 0.1 μm to about 50 μm.

According to the invention, it is preferred that apatite composition is in a particulate form, wherein the particles have an average size of about 10⁻³ to about 200 μm, more preferably about 2×10⁻³ μm to about 200 μm, even more preferably 5×10⁻² to about 150 μm, yet even more preferably about 0.1 μm to about 100 μm, yet even more preferably about 0.1 μm to about 80 μm and in particular about 0.1 μm to about 50 μm.

### Uses of the Apatite Composition

The apatite composition according to the present invention can advantageously be used as a dental filling material, a dental bonding cement, a bone cement and a bone replacing material. Additionally, it can advantageously be used in dental care and cleansing products, glass-ceramics, bone scaffolds and bone substrates, wherein the dental care product is preferably toothpaste.

Preferably, the dental care and cleansing products, glass-ceramics, bone scaffolds, bone substrates, toothpaste, gel, spray or liquid comprises fluorescin or a pharmaceutically acceptable salt thereof, preferably a metal salt, and more preferably a metal salt wherein the metal is selected from Group 1 or Group 2 of the Periodic System of the Elements for the detection of plaque. According to the invention, it is preferred that the amount of fluorescin of the pharmaceutically acceptable salt thereof that is comprised by the products mentioned above is between about 0.01 wt. % to about 20 wt %, preferably 0.1 wt. % to about 15 wt. %, based on the total eight of the product.

The apatite composition can further be used as a coating material and/or adhesive material for shaped dental articles selected from the group consisting of dental root posts, dental implants, bridges (three-section or multi-section), frames for bridges, (partial) crowns, inlays, onlays and joints, in particular zirconium-based shaped articles or shaped articles based on other porcelain-type materials. The zirconium-based shaped articles may be stabilised by the addition of other metal oxides such as yttrium oxide, hafnium oxide, aluminium oxide and mixtures thereof. Most preferably, the zirconium-based shaped articles comprise 2-10 mole percent of yttrium oxide (calculated as Y₂O₃), based on the total weight of the the zirconium-based shaped article.

Common glass ionomer based cements are known to bind chemically to teeth. When used as a cement for placing a crown, they provide a strong adhesion of the crown to the prepared tooth structure (which is essentially comprised of dentin). However, the glass ionomer based cements do not or hardly bond to the material of which the crown is made, e.g. zirconium oxide, which implies that they only provide a mechanical adhesion. However, the apatite composition according to the present invention is capable of forming a chemical bond between the crown and the apatite composition.

In particular, the inside of a crown, particularly a zirconium-based crown as described above, is coated with the apatite composition according to the present invention, and the crown is subsequently sintered in an oven. After application of the crown, a chemical bond is formed between the apatite of the tooth element upon which the crown is placed and the material from which the crown is made due to ion exchange. Hence, the present invention also relates to the use of the apatite composition as a coating material and/or adhesive material, wherein the inside of a crown is coated with the apatite composition followed by sintering the coated crown, where after the crown is applied to the tooth element. Accordingly, the present invention also relates to a process for applying a crown to a tooth element, wherein (a) the inside of the crown is coated with the apatite composition according to the present invention, (b) the coated crown is sintered and (c) the sintered crown is applied to the tooth element by using a cement comprising either the apatite composition according to the present invention or a cement comprising a convetional glass ionomer and a apatite, wherein the apatatite is selected from the fluoroapatite as defined above or a hydroxyapatite as defined above. It is well known that when the crown is sintered, its microstructure is compacted to about 99.5% of the theoretical density and that strength and toughness then reach high values as desired.

The present invention also relates to the use of the apatite composition as a coating material and/or adhesive material for adhering a crown to a tooth structure, wherein the tooth structure is first coated with the apatite composition according to the present invention followed by the placement of the crown.

The present invention also relates to the use of the apatite composition according to the present invention as a coating material and/or adhesive material for shaped dental articles selected from the group consisting of dental root posts, dental implants, bridges (three-section or multi-section), frames for bridges, (partial) crowns, inlays, onlays and joints.

The present invention also relates to the use of the apatite compositions according to the present invention for establishing a chemical bond (fusion) between ungrounded enamel or dentine surface and mineralisation of the material itself into fluor(hydroxy) apatite crystals bonded to Zirconia crown and bridge materials which are fired with a thin layer fluor/hydroxy apatite

### EXAMPLES

### Example 1

The following glass carbomer compositions were prepared according to the method disclosed in WO 20040108095 from the following ingredients:
(a) a polydimethylsiloxane having a kinematic viscosity of 1000 cSt (25° C.) indicated by S20;
(b) a conventional fluorosilicate glass powder; and
(c) a conventional aqueous polyacrylate solution.

The fluorosilicate glass powder and aqueous polyacrylate solution used to prepare the compositions were taken from A3 APLICAP capsules from 3M ESPE.

The amounts of the ingredients are listed in Table 1, wherein 5 wt. % additional fluorosilicate glass powder equals about 0.015 g fluorosilicate glass powder and wherein 0.0015 g S20 equals about 1.6 wt. % additional liquid added to the normal quantity of aqueous polyacrylate solution (about 0.0920 g).

TABLE 1

Composition relative to contents of
commercially available A3 APLICAP capsule

| | S20 | Additional fluorosilicate |
|---|---|---|
| Product | (g) | glass powder (wt. %) |
| 4P | 0.0015 | 5.00 |
| 5P | 0.0015 | 6.25 |
| 6P | 0.0015 | 7.50 |
| 8P | 0.0015 | 10.00 |
| 12P | 0.0045 | 15.00 |

### Example 2

The effect of the addition of 1 wt. % of the polysiloxane S20 (cf. WO 2004/108095, Example 1) to a commercially available fluorosilicate glass powder (indicated by the abbreviation FS 40; commercially available from Fuji) on flexural strength and elasticity modulus was investigated. In this experiment, the polysiloxane was mixed with the glass powder according to the procedure disclosed in Example 1 of WO 2004/108095 (in Example 1 the amount of polysiloxane was 1.6 wt. %). The products (with and without polysiloxane) were pressed to disks having a diameter of about 19 mm under a pressure provided by a weight of 5 kg during 0.30 s. It appeared that the addition of S20 increased the flexural strength and elasticity modulus. The data are shown in Table 2.

TABLE 2

| | Disk | Flexural | Elasticity |
|---|---|---|---|
| | diameter | Strength | Modulus |
| Glass powder | [mm] | [MPa] | [GPa] |
| FS 40 No. 1 (+PS) | 18.0 | 46 | 12.3 |
| FS 40 No. 1 (no PS) | 18.5 | 33 | 10.9 |
| FS 40 No. 2 (no PS) | 20.0 | 31 | 11.0 |

### Example 3

The solubility of the disks FS 40 No. 1 (+PS) and FS 40 No. 2 (no PS) were evaluated at physiological pH (cf. WO 2004/108095, Example 3). In particular in the period of the first 10 minutes after fillings have been made, they are very vulnerable for degradation by aqueous compositions, e.g. saliva. The solubility data are shown in FIG. 1 and they show that the glass carbomer composition shows less degradation.

### Example 4

The compositions according to the present invention are prepared by conventional means. Fluoroapatite glass powder is milled to particles having the desired average size of about 10⁻³ to about 200 μm. Optionally, the fluoroapatite glass powder is mixed with other glass powders, e.g. hydroxyapatite, Ca-glass and/or Sr-glass. In a next step, the fluoroapatite glass powder or the mixture of the fluoroapatite glass powder and other glass powders are mixed with a glass carbomer composition in a desired ratio.

These compositions were analysed by XRD (using a Philips Powder Diffractometer PW 1700 series with a copper (Cu-Kα) x-ray source; powder samples (average particle size <45 μm) were scanned between 2θ=5-80° with a step size of 2θ=0.04) and by MAS-NMR spectroscopy (Bruker 200 MHz; for ¹⁹F: spinning rate=10 kHz, recycle time 10 s; for ³¹P: spinning rate=3 kHz, recycle time 20 s).

The XRD-analysis revealed that the compositions were completely amorphous.

MAS-NMR spectroscopy revealed that the compositions comprise nanocrystals of calcium fluoroapatite which act as nuclei for the remineralisation process. The compositions have a much smaller average particle size than in conventional glass ionomer cements.

While normal calcium fluoroapatite show in the ¹⁹F MAS-NMR spectrum a significant sharp resonance of F-Ca(3) at −103 ppm, this resonance is practically absent in the compositions according to the invention.

In the ³¹P MAS-NMR spectrum, a significant orthophosphate resonance (originating from the calcium fluoroapatite; 3-4 ppm) and a significant pyrophosphate resonance was observed.

1-31. (canceled)

32. An apatite composition comprising 0.001-99.999 wt % of an apatite and 0.001-99.999 wt. % of a glass carbomer, based on the total weight of the apatite composition, wherein the glass carbomer is obtainable by treating a fluorosilicate glass powder with:
(a) a poly(dialkylsiloxane) having terminal hydroxyl groups, wherein the alkyl groups contain 1 to 4 carbon atoms,
(b) an aqueous acid solution,
and separating the treated fluorosilicate glass powder from the aqueous acid solution.

33. The apatite composition according to claim 32, wherein the apatite is a fluoroapatite.

34. The apatite composition according to claim 33, wherein the fluoroapatite has the general formula (I):
Ca₁₀₋ₓMₓ(PO₄)_{6-y}B_{y}A_{z}(OH)_{2-z} (I)
wherein:
M is a cation other than Ca²⁺;
B is an anion other than PO₄³⁻;
A is F⁻;
0≦x≦9;
0≦y≦5; and
0≦z≦2.

35. The apatite composition according to claim 34, wherein x=y=0 and z=2.

36. The apatite composition according to of claim 33, further comprising an apatite according to the general formula (II):
Ca₁₀₋ₓMₓ(PO₄)_{6-y}B_{y}A_{z}(OH)_{2-z} (II)
wherein:
M is a cation other than Ca²⁺;
B is an anion other than PO₄³⁻;
A is O²⁻, CO₃²⁻, Cl⁻, OH⁻ and mixtures thereof;
0≦x≦9;
0≦y≦5; and
0≦z≦2.

37. The apatite composition according to claim 36, wherein x=y=z=0 and A is OH⁻.

38. The apatite composition according to claim 32, wherein the poly(dialkylsiloxane) is linear or cyclic.

39. The apatite composition according to claim 38, wherein the alkyl groups of the poly(dialkylsiloxane) are methyl groups.

40. The apatite composition according to claim 38, wherein the poly(dialkylsiloxane) has a kinematic viscosity in the range of about 1 to about 100,000 cSt at 25° C.

41. The apatite composition according to claim 32, wherein the particles of the fluorosilicate glass powder have an average size of about 10⁻³ to about 200 μm.

42. The apatite composition according to claim 32, wherein the aqueous acid solution comprises an inorganic acid or an organic acid.

43. The apatite composition according to claim 32, wherein the fluoroapatite composition is in a particulate form.

44. The apatite composition according to claim 43, wherein the particles of the fluoroapatite composition have an average size of about 10⁻³ to about 200 μm.

45. A dental filling material comprising the apatite composition according to claim 32.

46. A denting bonding cement comprising the apatite composition according to claim 32.

47. A bone cement comprising the apatite composition according to claim 32.

48. A bone replacing material comprising the apatite composition according to claim 32.

49. A dental care product comprising the apatite composition according to claim 32.

50. A dental care product according to claim 49, wherein the dental care product is a toothpaste, gel, spray or liquid.

51. A cleansing product comprising the apatite composition according to claim 32.

52. A glass-ceramic material comprising the apatite composition according to claim 32.

53. A bone scaffold material comprising the apatite composition according to claim 32.

54. A bone substrate comprising the apatite composition according to claim 32.

55. A coating material and/or an adhesive material for shaped dental articles selected from the group consisting of dental root posts, dental implants, bridges (three-section or multi-section), frames for bridges, (partial) crowns, inlays, onlays and joints, wherein the coating material and/or the adhesive material comprises the apatite composition according to claim 32.
